(19) United States
(12) Patent Application Publication
(10) Pub. No.: US 2004/0136932 A1
(43) Pub. Date: Jul. 15, 2004

(54) Photoprotective cosmetic compositions comprising dibenzoylmethane compounds and 3-(2-azacycloalkylidene)-1,3-dihydroindol-2-one compounds

(75) Inventors: Roger Rozot, Lagny/Marne (FR); Andre Deflandre, Orry La Ville (FR)

Correspondence Address: BURNS DOANE SWECKER & MATHIS L L P, POST OFFICE BOX 1404, ALEXANDRIA, VA, 22313-1404, US

(73) Assignee: SOCIETE L'OREAL S.A., Paris (FR)

(21) Appl. No.: 10/720,174

(22) Filed: Nov. 25, 2003

Related U.S. Application Data
(60) Provisional application No. 60/444,948, filed on Feb. 5, 2003.

(30) Foreign Application Priority Data
Nov. 29, 2002 (FR) 02/15057

(51) Int. Cl. A61K 7/42; C 07D 4 3/02

(52) U.S. Cl. 424/59; 540/596; 546/201; 548/465

(57) ABSTRACT

Topically applicable cosmetic/dermatological sunscreen compositions well suited for the UV-photoprotection of the skin and/or hair, contain (a) an effective UV-screening amount of at least one dibenzoylmethane compound and (b) an effective UV-photostabilizing amount of at least one 3-(2-azacycloalkylidene)-1,3-dihydroindol-2-one compound, formulated into (c) a topically applicable, cosmetically/dermatologically acceptable carrier therefor.

### CROSS-REFERENCE TO PRIORITY/PROVISIONAL APPLICATIONS

This application claims priority under 35 U.S.C. § 119 of FR-02/15057, filed Nov. 29, 2002, and of provisional application Serial No. 60/444,948, filed Feb. 5, 2003, both hereby expressly incorporated by reference. This application is also a continuation of said '948 provisional.

### CROSS-REFERENCE TO COMPANION APPLICATION

Copending application Ser. No. ______ [Attorney Docket No. 016800-585], filed concurrently herewith and assigned to the assignee hereof.

### BACKGROUND OF THE INVENTION

1. Technical Field of the Invention

The invention relates to cosmetic or dermatological compositions for topical application, in particular for photoprotecting the skin and the hair against UV radiation, which comprise, in a cosmetically acceptable support:

(a) at least one UV-screening agent of the type derived from dibenzoylmethane, and

(b) at least one 3-(2-azacycloalkylidene)-1,3-dihydro-indol-2-one derivative in an effective amount to photostabilize said dibenzoylmethane derivative.

2. Description of Background/Related/Prior Art

It is known that light radiation with wavelengths of between 280 nm and 400 nm permits tanning of the human epidermis and that light rays with wavelengths more particularly between 280 nm and 320 nm, known as UV-B rays, cause skin burns and erythema which can harm the development of a natural tan. For these reasons, as well as for aesthetic reasons, there is a constant demand for means of controlling this natural tanning in order thus to control the color of the skin; this UV-B radiation should thus be screened out.

It is also known that UV-A rays, with wavelengths between 320 nm and 400 nm, which cause tanning of the skin, are liable to induce adverse changes therein, in particular in the case of sensitive skin or skin which is continually exposed to solar radiation. UV-A rays cause in particular a loss of elasticity of the skin and the appearance of wrinkles leading to premature ageing of the skin. They promote triggering of the erythemal reaction or amplify this reaction in certain individuals and may even be the cause of phototoxic or photoallergic reactions. Thus, for aesthetic and cosmetic reasons such as the conservation of the skin's natural elasticity, for example, an increasingly large number of people wish to control the effect of UV-A rays on their skin. It is thus desirable also to screen out UV-A radiation.

In this respect, one particularly advantageous family of UV-A screening agents currently consists of dibenzoylmethane derivatives, and in particular 4-tert-butyl-4′-methoxydibenzoylmethane, which have high intrinsic absorbing power. These dibenzoylmethane derivatives, which are products which are now well known per se as screening agents that are active in the UV-A range, are described in particular in FR-A-2,326,405 and FR-A-2,440,933, as well as in EP-A-0,114,607; 4-tert-butyl-4′-methoxy-dibenzoylmethane is moreover currently sold under the trademark “Parsol 1789” by Hoffmann LaRoche.

Unfortunately, it is found that dibenzoyl-methane derivatives are products that are relatively sensitive to ultraviolet radiation (especially UV-A), i.e., more specifically, they have an annoying tendency to be degraded more or less quickly under the action of this UV. Thus, this substantial lack of photochemical stability of dibenzoylmethane derivatives towards ultraviolet radiation, to which they are by nature intended to be subjected, does not make it possible to ensure constant protection during prolonged exposure to the sun, and so the user must make repeated applications at regular and close time intervals in order to obtain effective protection of the skin against UV rays.

The photostabilization of dibenzoylmethane derivatives with respect to UV radiation is, at the present time, a problem that has still not been solved entirely satisfactorily.

### SUMMARY OF THE INVENTION

It has now surprisingly and unexpectedly been determined that by combining an effective amount of a 3-(2-azacycloalkylidene)-1,3-dihydroindol-2-one compound with the dibenzoylmethane compounds indicated above, it is possible to substantially and appreciably improve the photochemical stability (or photostability) of these same dibenzoylmethane derivatives.

This essential discovery forms the basis of the present invention.

### DETAILED DESCRIPTION OF BEST MODE AND SPECIFIC/PREFERRED EMBODIMENTS OF THE INVENTION

Thus, the present invention features a novel cosmetic or dermatological compositions, for topical application, which comprise, in a cosmetically acceptable support:

(a) at least one UV-screening agent of the type derived from dibenzoylmethane, and

(b) at least one 3-(2-azacycloalkylidene)-1,3-dihydro-indol-2-one derivative in an effective photostabilizing amount.

The present invention also features a process for improving the stability of at least one dibenzoylmethane compound with respect to UV radiation, which entails intimately admixing said dibenzoylmethane compound with an effective photostabilizing amount of at least one 3-(2-azacycloalkylidene)-1,3-dihydroindol-2-one compound.

For the purposes of the present invention, the expression “effective amount of a 3-(2-azacyclo-alkylidene)-1,3-dihydroindol-2-one derivative” means an amount that is sufficient to obtain an appreciable and significant improvement in the photostability of the dibenzoylmethane derivative(s) of the photoprotective cosmetic composition. This minimum amount of photostabilizer to be used, which may vary depending on the nature of the cosmetically acceptable support selected for the composition, may be determined without any difficulty by means of a standard test for measuring photostability.

For the purposes of the present invention, the expression “3-(2-azacycloalkylidene)-1,3-dihydro-indol-2-one derivative” means any simple, oligomeric or polymeric compound containing on the chain grafts containing the 3-(2-azacycloalkylidene)-1,3-dihydro-indol-2-one group.

Finally, this invention also features the use of at least one 3-(2-azacyclo-alkylidene)-1,3-dihydroindol-2-one compound in the preparation of a cosmetic or dermatological composition comprising at least one dibenzoylmethane compound, with the aim of improving the stability towards UV rays of said dibenzoylmethane compound.

Other characteristics, aspects and advantages of the present invention will emerge on reading the detailed description that follows.

The 3-(2-azacycloalkylidene)-1,3-dihydro-indol-2-one compounds in accordance with the invention are preferably selected from those corresponding to the general formula (I) below:

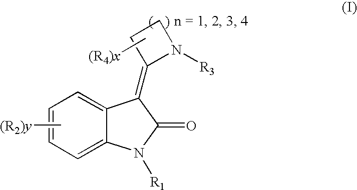

in which: R₁ and R₃, which may be identical or different, are each a hydrogen atom, a linear or branched, saturated or unsaturated C₁-C₂₂ alkyl radical optionally substituted with one or more groups A₁, a saturated or unsaturated ring member of 4 to 7 atoms, optionally containing at least one heteroatom selected from among N, O and S, optionally fused with another ring, optionally substituted with one or more groups A₁, with the proviso that these rings may contain one or more carbonyl or thiocarbonyl functions, one of the groups C(═NR₅)R′₅, C(═NR₅)NR′₅R″₅, COR₅, CSR₅, COOR₅, CONR₅R′₅, CSNR₅R′₅, SO₂R₅ and SO₂NR₅R′₅ wherein R₅, R′₅ and R″₅, which may be identical or different, are each hydrogen, a linear or branched C₁-C₂₂ alkyl radical or a ring member of 4 to 7 atoms, optionally containing at least one heteroatom selected from among N, O and S, optionally fused with another ring, with the proviso that these rings mat contain one or more carbonyl or thiocarbonyl functions, the alkyl radical of said rings being saturated or unsaturated and optionally substituted with at least one substituent A₂; R₂ and R₄, which may be identical or different, are each a hydrogen atom, a linear or branched, saturated or unsaturated C₁-C₂₂ alkyl radical optionally substituted with one or more groups A₁, a saturated or unsaturated ring member of 4 to 7 atoms, optionally containing at least one heteroatom selected from among N, O and S, optionally substituted with one or more groups A₁, optionally fused with another ring or with another 3-(2-azacyclo-alkylidene)-1,3-dihydroindol-2-one according to this invention, with the proviso that these rings may contain one or more carbonyl or thiocarbonyl functions, optionally positively charged like imidazolium, pyridinium, pyrazolium or triazolium, a halogen such as F, Cl or Br, one of the groups CF₃, CN, OR₅, SR₅, NR₅R′₅, C(═NR₅)R₁₅, C(═NR₅)NR′₅R″₅, NR₅C(═NR′₅)NR″₅R′″₅, COR₅, CSR₅, COOR₅, CONR₅R′₅, NR₅COR′₅, NR₅CONR′₅R″₅, CSNR₅R′₅, SO₂NR₅R′₅, NR₅SO₂R′₅, SO₂R₅ and NR₅R′₅R″₅R′″₅+ wherein R₅, R′₅, R″₅ and R′″₅, which may be identical or different, are each hydrogen, a linear or branched C₁-C₂₂ alkyl radical or a ring member of 4 to 7 atoms, which may contain at least one heteroatom selected from among N, O and S, optionally fused with another ring, with the proviso that these rings may contain one or more carbonyl or thiocarbonyl functions, the alkyl radical or the said rings being saturated or unsaturated and optionally substituted with at least one substituent A₂; A₁ is a halogen such as F, Cl or Br, a linear or branched, saturated or unsaturated C₁-C₂₂ alkyl radical, optionally substituted with one or more groups A₂, one of the groups CF₃, CN, OR₅, SR₅, NR₅R′₅, C(═NR₅)R′₅, C(═NR₅)NR′₅R″₅, NR₅C(═NR′₅)NR″₅R′″₅, COR₅, CSR₅, COOR₅, CONR₅R′₅, NR₅COR′₅, NR₅CONR′₅R″₅, CSNR₅R′₅, SO₂NR₅R′₅, NR₅SO₂R′₅, SO₂R₅, SiR₅R′₅R″₅, SiR₅(OSiR′₅R″₅R′″₅)OSiR′₅R″₅R′″₅ and NR₅R′₅R″₅R′″₅+wherein R₅, R′₅, R″₅ and R′″₅, which may be identical or different, are each hydrogen, a linear or branched C₁-C₂₂ alkyl radical or a ring member of 4 to 7 atoms, which may contain at least one heteroatom selected from among N, O and S, optionally fused with another ring, with the proviso that these rings may contain one or more carbonyl or thiocarbonyl functions, the alkyl radical or said rings being saturated or unsaturated and optionally substituted with at least one substituent A₂, a saturated or unsaturated ring member of 4 to 7 atoms, optionally containing at least one heteroatom selected from among N, O and S, optionally substituted with one or more groups A₂, optionally fused with another ring or with another 3-(2-azacyclo-alkylidene)-1,3-dihydroindol-2-one according to this invention, with the proviso that these rings may contain one or more carbonyl or thiocarbonyl functions, optionally positively charged like imidazolium, pyridinium, pyrazolium or triazolium, A₂ is a halogen such as F, Cl or Br, a linear or branched, saturated or unsaturated C₁-C₂₂ alkyl radical, one of the groups CF₃, CN, OR, SR, NRR′, C(═NR)R′, C(═NR)NR′R″, NRC(═NR′)NR″R′″, COR, CSR, COOR, CONRR′, NRCOR′, NRCONR′R″, CSNRR′, SO₂NRR′, NRSO₂R′, SO₂R, SiRR′R″, SiR(OSiR′R″R′″)OSiR′R″R′″ and NRR′R″R′″+wherein R, R′, R″ and R′″, which may be identical or different, are each hydrogen or a linear or branched C₁-C₂₂ alkyl radical; y is 1, 2, 3 or 4; and x ranges from 1 to 2n+2.

As examples of linear or branched, saturated or unsaturated C₁-C₂₂ alkyl radicals, mention may be made of: methyl, isopropyl, 2-ethylhexyl, tert-butyl, ethylene, propylene. This list is not limiting.

Examples of heterocycles that may be mentioned include: pyrrole, furan, thiophene, imidazole, oxazole, thiazole, pyrazole, isoxazole, isothiazole, triazole, oxadiazole, thiadiazole, tetrazole, pyridine, piperidine, pyrimidine, piperazine, pyridazine, pyrazine, triazine, morpholine, pyrrolidine, thiazolidine. This list is not limiting.

Examples of saturated or unsaturated carbocycles containing 4, 5, 6 or 7 atoms that may be mentioned include: cyclobutyl, cyclopentyl, cyclohexyl, cyclohexenyl, phenyl, cycloheptyl. This list is not limiting.

Certain 3-(2-azacycloalkylidene)-1,3-dihydroindol-2-one compounds of formula (I) in accordance with the invention are known in the chemical literature and in particular certain of them have been described in the articles in Indian Journal of Chemistry (1983), 22B, 1083-1086 and in Journal of Medicinal Chemistry (1989), 32, 437-444.

The 3-(2-azacycloalkylidene)-1,3-dihydro-indol-2-one compounds of formula (I) in accordance with the invention may be prepared according to a synthetic process using a lactam acetal and a 1,3-di-hydroindol-2-one derivative at room temperature in an anhydrous ether according to the reaction scheme below cited in the above articles:

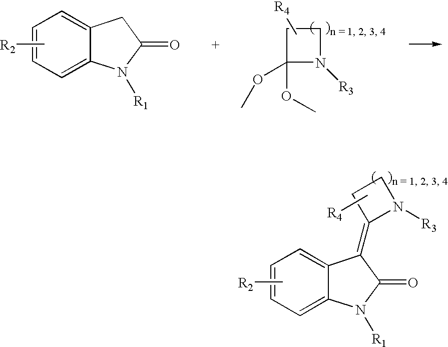

Many lactam and 1,3-dihydroindol-2-one derivatives are available from most of the chemical product suppliers, for instance:

| | Supplier | Reference | CAS |
|---|---|---|---|
| 1,3-dihydroindol-2- | | | |
| one Derivative | | | |
| 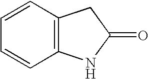 | ABCR | AV18764 | 59-48-3 |
| 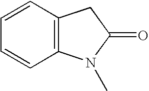 | Maybridge | RH 01320 | 61-70-1 |
| 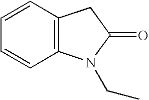 | MicroChemistry Ltd. | 39248 | 61-28-9 |
| 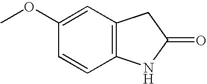 | Specs | AK- 830/25033062 | |
| 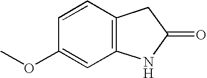 | Finoraga SA | 000048 | |
| 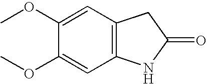 | Maybridge | SEW 04570 | 6286-64-2 |
| 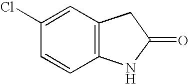 | Aldrich | 12,748-5 | 17630-75-0 |
| 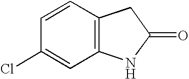 | ABCR | AV9674 | 56341-37-8 |
| 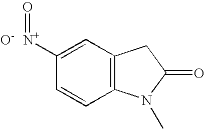 | Maybridge | RH 01323 | 20870-89-7 |
| 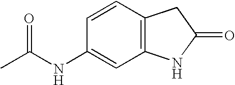 | MDPI | 11566 | |
| 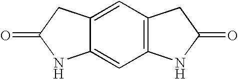 | MDPI | 11349 | |
| 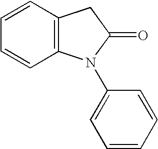 | Specs | AC- 907/25005257 | |
| 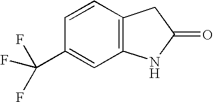 | Butt Park Ltd. | 24/07-32 | |
| Lactam Derivative | | | |
| 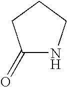 | ABCR | AV15332 | 616-45-5 |
| 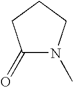 | ABCR | AV12260 | 872-50-4 |
| 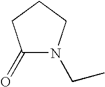 | ABCR | 1549765 | 2687-91-4 |
| 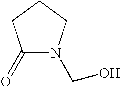 | Salor | S36,255-7 | 15438-71-8 |
| 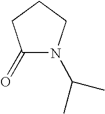 | BASF-Misc. | | 3772-26-7 |
| 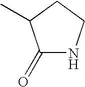 | Lancaster | | 2555-05-7 |
| 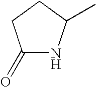 | Aldrich | M7,970-0 | 108-27-0 |
| 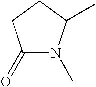 | Aldrich | D18,410-1 | 5075-92-3 |
| 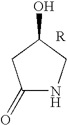 | Aldrich | 47,916-0 | 22677-21-0 |
| 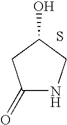 | Aldrich | 47,917-9 | 68108-18-9 |
| 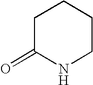 | ABCR | AV12271 | 675-20-7 |
| 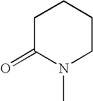 | Advan-Synth | 001427 | 931-20-4 |
| 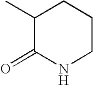 | Maybridge | BTBG 00104 | |
| 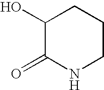 | Salor | S43,760-3 | 19365-08-3 |
| 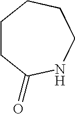 | ABCR | AV19374 | 105-60-2 |
| 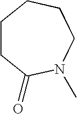 | ABCR | AV17776 | 2556-73-2 |
| 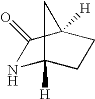 | Acros | 29704-0010 | 79200-56-9 |
| 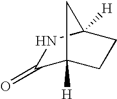 | Acros | 29705-0010 | 130931-83-8 |
| 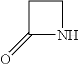 | Aldrich | 32,846-4 | 930-21-2 |
| 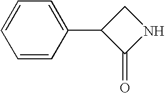 | Salor | S90,675-1 | 17197-57-8 |
| 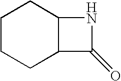 | Acros | 33584-0010 | 34102-49-3 |
| 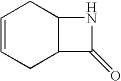 | Maybridge | JFD 02755 | |
| 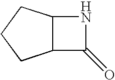 | Acros | 33583-5000 | 22031-52-3 |

The lactam acetals may be obtained from lactams according to the reaction scheme below cited in the articles Tetrahedron Letters (1994), 35 (18), 2951-2954 and Journal of Organic Chemistry (1984), 49, 3659-3660:

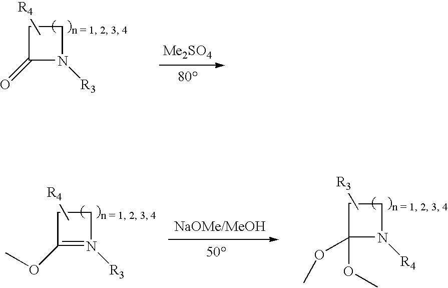

Depending on the starting lactam, steps of protection and then of deprotection of certain functional groups such as OH or NH, which are well known to those skilled in the art, may be necessary.

Among the preferred compounds of formula (I) that may be mentioned are those of formula (II) or (III) below:

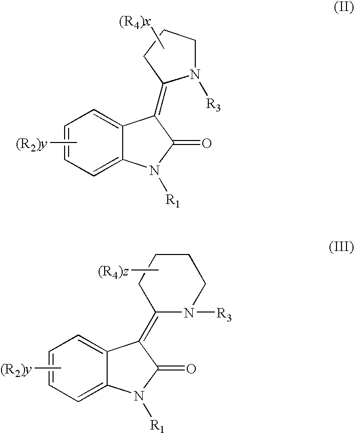

in which R₁ and R₃, which may be identical or different, are each hydrogen, a linear or branched, saturated or unsaturated C₁-C₈ alkyl radical, a phenyl radical, or a radical COR₅ or SO₂R₅ wherein R₅ is a linear or branched, saturated or unsaturated C₁-C₈ alkyl radical or a phenyl group; R₂ is hydrogen; a linear or branched, saturated or unsaturated C₁-C₈ alkyl radical; a phenyl group; one of the radicals OR₅, NR₅R′₅, NR₅COR′₅, COOR₅ and CONR₅R′₅ wherein R₅ and R′₅, which may be identical or different, are each hydrogen, a linear or branched, saturated or unsaturated C₁-C₈ alkyl radical, a phenyl radical, or the CF₃ radical; R₄ is hydrogen, a linear or branched, saturated or unsaturated C₁-C₈ alkyl radical, a phenyl radical or a radical OR₅ wherein R₅ is hydrogen, a linear or branched, saturated or unsaturated C₁-C₈ alkyl radical, or a phenyl radical; and x, y and z are each 1, 2, 3 or 4.

Among the 3-(2-azacycloalkylidene)-1,3-dihydroindol-2-one compounds of formula (I) that will be mentioned even more particularly are those of formula (II) in which:

R₂ denotes hydrogen, OR₅, NR₅COR′₅ or NR₅R′₅ with R₅ and R′₅, which may be identical or different, denoting hydrogen, a linear or branched, saturated or unsaturated C₁-C₈ alkyl radical or a phenyl group;

R₄ denotes hydrogen; R₁ and R₃, which may be identical or different, denote hydrogen, a linear or branched, saturated or unsaturated C₁-C₈ alkyl radical, or a phenyl group, and even more particularly those described in the table below, which are commercially available from Specs/Biospecs:

| Compound | | Name (Specs/Biospecs |
|---|---|---|
| No. | Formula | Reference) |
| 1 | 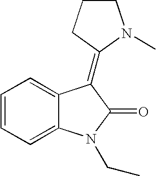 1-Ethyl-3-(1-methyl- pyrrolidin-2-ylidene)-1,3- dihydroindol-2-one (AH-262/33341016) | |
| 2 | 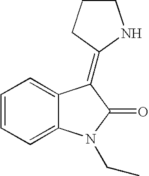 | 1-Ethyl-3-pyrrolidin-2- ylidene-1,3-dihydro- indol-2-one (AH-262/33341015) |
| 3 | 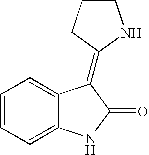 | 3-Pyrrolidin-2-ylidene-1,3- dihydroindol-2-one (AH-262/33341017) |

The 3-(2-azacycloalkylidene)-1,3-dihydro-indol-2-one derivatives in accordance with the invention are preferably present in the composition of the invention in proportions ranging from 0.01% to 20% by weight and more preferably from 0.1% to 10% by weight relative to the total weight of the composition.

As mentioned previously, the dibenzoylmethane derivatives intended to be photostabilized in the context of the present invention are products that are already well known per se and described especially in the above-mentioned documents FR-2,326,405, FR-2,440,933 and EP-0,114,607, the teachings of which documents are, for that which concerns the actual definition of these products, entirely included in the present description by reference.

According to the present invention, it is quite clearly possible to use one or more dibenzoyl-methane derivatives.

Among the dibenzoylmethane derivatives that particularly fall within the context of the present invention, mention may be made especially, in a non-limiting manner, of:

2-methyldibenzoylmethane,

4-methyldibenzoylmethane,

4-isopropyldibenzoylmethane,

4-tert-butyldibenzoylmethane,

2,4-dimethyldibenzoylmethane,

2,5-dimethyldibenzoylmethane,

4,4′-diisopropyldibenzoylmethane,

4,4′-dimethoxydibenzoylmethane,

4-tert-butyl-4′-methoxydibenzoylmethane,

2-methyl-5-isopropyl-4′-methoxydibenzoylmethane,

2-methyl-5-tert-butyl-4′-methoxydibenzoylmethane,

2,4-dimethyl-4′-methoxydibenzoylmethane,

2,6-dimethyl-4-tert-butyl-4′-methoxydibenzoylmethane.

Among the dibenzoylmethane derivatives mentioned above, it is most particularly preferred, according to the present invention, to use 4-tert-butyl-4′-methoxydibenzoylmethane, especially the product sold under the trademark “Parsol 1789” by Hoffmann LaRoche, this screening agent thus corresponding to the following structural formula (or to one of its tautomeric forms):

Another dibenzoylmethane derivative that is preferred according to the present invention is 4-iso-propyldibenzoylmethane, a screening agent sold under the name “Eusolex 8020” by Merck, and corresponding to the following structural formula (or to one of its tautomeric forms):

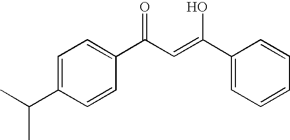

The dibenzoylmethane derivative(s) may be present in the compositions in accordance with the invention in contents preferably ranging from 0.01% to 20% by weight and more preferably from 0.1% to 10% by weight relative to the total weight of the composition.

The compositions in accordance with the invention may also comprise other additional UVA-active and/or UVB-active organic or mineral UV-screening agents that are water-soluble or liposoluble or insoluble in the cosmetic solvents commonly used.

The additional organic screening agents are chosen especially from anthranilates; cinnamic derivatives; salicylic derivatives; camphor derivatives; triazine derivatives such as those described in U.S. Pat. No. 4,367,390, EP-863,145, EP-517,104, EP-570,838, EP-796,851, EP-775,698, EP-878,469, EP-933,376, EP-507,691, EP-507,692, EP-790,243, EP-944,624; benzophenone derivatives; p,p-diphenylacrylate derivatives; benzotriazole derivatives; benzalmalonate derivatives; benzimidazole derivatives; imidazolines; bis-benzazolyl derivatives as described in EP-669,323 and U.S. Pat. No. 2,463,264; p-aminobenzoic acid (PABA) derivatives; methylenebis(hydroxyphenylbenzotriazole) derivatives as described in U.S. Pat. Nos. 5,237,071, 5,166,355, GB-2,303,549, DE-197,26,184 and EP-893,119; benzoxazole derivatives such as those described in the EP-O-832,642, EP-1-027,883, EP-1-300,137 and DE-101,62,844; the screening polymers and screening silicones such as those described especially in WO 93/04665; dimers derived from α-alkylstyrene, such as those described in DE-198,55,649; 4,4-diarylbutadienes such as those described in EP-O-967,200, DE-197,46,654, DE-197,55,649, EP-A-1,008,586, EP-1-133,980 and EP-133,981, and mixtures thereof.

As examples of additional organic screening agents, mention may be made of those denoted hereinbelow under their INCI name:

para-Aminobenzoic Acid Derivatives:

PABA,

Ethyl PABA,

Ethyl dihydroxypropyl PABA,

Ethylhexyl dimethyl PABA sold in particular under the name “Escalol 507” by

ISP,

Glyceryl PABA,

PEG-25 PABA sold under the name “Uvinul P25” by BASF.

Salicylic Derivatives:

Homosalate sold under the name “Eusolex HMS” by Rona/EM Industries,

Ethylhexyl salicylate sold under the name “Neo Heliopan OS” by Haarmann and Reimer,

Dipropylene glycol salicylate sold under the name “Dipsal” by Scher,

TEA salicylate sold under the name “Neo Heliopan TS” by Haarmann and Reimer.

Cinnamic Derivatives:

Ethylhexyl methoxycinnamate sold in particular under the trademark “Parsol MCX” by Hoffmann LaRoche,

Isopropyl methoxycinnamate,

Isoamyl methoxycinnamate sold under the trademark “Neo Heliopan E 1000” by

Haarmann and Reimer,

Cinoxate,

DEA methoxycinnamate,

Diisopropyl methylcinnamate,

Glyceryl ethylhexanoate dimethoxycinnamate.

β,β′-Diphenylacrylate Derivatives:

Octocrylene sold in particular under the trademark “Uvinul N539” by BASF,

Etocrylene sold in particular under the trademark “Uvinul N35” by BASF.

Benzophenone Derivatives:

Benzophenone-1 sold under the trademark “Uvinul 400” by BASF,

Benzophenone-2 sold under the trademark “Uvinul D50” by BASF,

Benzophenone-3 or Oxybenzone sold under the trademark “Uvinul M40” by BASF,

Benzophenone-4 sold under the trademark “Uvinul MS40” by BASF,

Benzophenone-5,

Benzophenone-6 sold under the trademark “Helisorb 11” by Norquay,

Benzophenone-8 sold under the trademark “Spectra-Sorb UV-24” by American Cyanamid,

Benzophenone-9 sold under the trademark “Uvinul DS-49” by BASF,

Benzophenone-12

n-hexyl 2-(4-diethylamino-2-hydroxybenzoyl)benzoate.

Benzylidenecamphor Derivatives:

3-Benzylidenecamphor manufactured under the name “Mexoryl SD” by Chimex,

4-Methylbenzylidenecamphor sold under the name “Eusolex 6300” by Merck,

Benzylidenecamphorsulphonic acid manufactured under the name “Mexoryl SL” by Chimex,

Camphor benzalkonium methosulphate manufactured under the name “Mexoryl SO” by Chimex,

Terephthalylidenedicamphorsulphonic acid manufactured under the name “Mexoryl SX” by Chimex,

Polyacrylamidomethylbenzylidenecamphor manufactured under the name “Mexoryl SW” by Chimex.

Phenylbenzimidazole Derivatives:

Phenylbenzimidazolesulphonic acid sold in particular under the trademark “Eusolex 232” by Merck,

Disodium phenyl dibenzimidazole tetrasulphonate, sold under the trademark “Neo Heliopan AP” by Haarmann and Reimer.

Triazine Derivatives:

Anisotriazine sold under the trademark “Tinosorb S” by Ciba Geigy,

Ethylhexyltriazone sold in particular under the trademark “Uvinul T150” by BASF,

2,4,6-tris(diisobutyl 4′-aminobenzalmalonate)-s-triazine,

Diethylhexylbutamidotriazone sold under the trademark “Uvasorb HEB” by Sigma 3V.

Phenylbenzotriazole Derivatives:

Drometrizole trisiloxane sold under the name “Silatrizole” by Rhodia Chimie,

Methylenebis(benzotriazolyl)tetramethylbutylphenol sold in solid form under the trademark “MIXXIM BB/100” by Fairmount Chemical, or in micronized form as an aqueous dispersion under the trademark “Tinosorb M” by Ciba Specialty Chemicals.

Anthranilic Derivatives:

Menthyl anthranilate sold under the trademark “Neo Heliopan MA” by Haarmann and Reimer.

Imidazoline Derivatives:

Ethylhexyldimethoxybenzylidenedioxoimidazoline propionate.

Benzalmalonate Derivatives:

Polyorganosiloxane containing benzalmalonate functions, as the product Polysilicone-15 sold under the trademark “Parsol SLX” by Hoffmann LaRoche

4,4-Diarylbutadiene Derivatives:

1,1-Dicarboxy(2,2′-dimethylpropyl)-4,4-diphenyl-butadiene,

Benzoxazole Derivatives:

2,4-bis-[5-1 (dimethylpropyl)benzoxazol-2-yl-(4-phenyl)-imino]-6-(2-ethylhexyl)-imino-1,3,5-triazine sold under the trademark Uvasorb K2A by Sigma 3V; and mixtures thereof.

The preferred additional organic UV-screening agents are chosen from:

Ethylhexyl salicylate,

Ethylhexyl methoxycinnamate,

Octocrylene,

Phenylbenzimidazolesulphonic acid,

Benzophenone-3,

Benzophenone-4,

Benzophenone-5,

n-Hexyl 2-(4-diethylamino-2-hydroxybenzoyl)-benzoate,

4-Methylbenzylidenecamphor,

Terephthalylidenedicamphorsulphonic acid,

Disodium phenyldibenzimidazoletetrasulphonate,

2,4,6-Tris(diisobutyl 4′-aminobenzalmalonate)-s-triazine,

Anisotriazine,

Ethylhexyltriazone,

Diethylhexylbutamidotriazone,

Methylenebis(benzotriazolyl)tetramethylbutyl-phenol,

Drometrizole trisiloxane,

Polysilicone-15

1,1-Dicarboxy(2,2′-dimethylpropyl)-4,4-diphenyl-butadiene,

2,4-bis-[5-1 (dimethylpropyl)benzoxazol-2-yl-(4-phenyl)-imino]-6-(2-ethylhexyl)-imino-1,3,5-triazine and mixtures thereof.

The additional mineral screening agents are chosen from pigments or nanopigments (mean size of the primary particles: generally between 5 nm and 100 nm and preferably between 10 nm and 50 nm) of coated or uncoated metal oxides such as, for example, nanopigments of titanium oxide (amorphous or crystallized in rutile and/or anatase form), of iron oxide, of zinc oxide, of zirconium oxide or of cerium oxide, which are all UV photoprotective agents that are well known per se. Standard coating agents are, moreover, alumina and/or aluminum stearate. Such coated or uncoated metal oxide nanopigments are described in particular in EP-518,772 and EP-518,773.

The additional UV-screening agents in accordance with the invention are generally present in the compositions according to the invention in proportions ranging from 0.1% to 20% by weight relative to the total weight of the composition, and preferably ranging from 0.2% to 15% by weight relative to the total weight of the composition.

The cosmetic compositions according to the invention may also contain agents for artificially tanning and/or browning the skin (self-tanning agents) such as dihydroxyacetone (DHA).

The compositions in accordance with the present invention may also comprise standard cosmetic adjuvants chosen especially from fatty substances, organic solvents, ionic or nonionic thickeners, softeners, humectants, antioxidants, moisturizers, desquamating agents, free-radical scavengers, antipollution agents, antibacterial agents, anti-inflammatory agents, depigmenting agents, propigmenting agents, opacifiers, stabilizers, emollients, silicones, antifoams, insect repellents, fragrances, preserving agents, anionic, cationic, nonionic, zwitterionic or amphoteric surfactants, substance P antagonists, substance CGRP antagonists, fillers, pigments, polymers, propellants, acidifying or basifying agents, or any other ingredient usually used in cosmetics and/or dermatology.

The fatty substances may consist of an oil or a wax or mixtures thereof. The term “oil” means a compound that is liquid at room temperature. The term “wax” means a compound that is solid or substantially solid at room temperature, and whose melting point is generally above 35° C.

Oils that may be mentioned include mineral oils (paraffin); plant oils (sweet almond oil, macadamia oil, blackcurrant seed oil or jojoba oil); synthetic oils, for instance perhydrosqualene, fatty alcohols, fatty acids or fatty acid esters (for instance the C₁₂-C₁₅ alcohol benzoate sold under the trademark “Finsolv TN” by Witco, octyl palmitate, isopropyl lanolate, triglycerides, including capric/caprylic acid triglycerides), oxyethylenated or oxypropylenated fatty esters and ethers; silicone oils (cyclomethicone and polydimethylsiloxanes, or PDMS) or fluoro oils, and polyalkylenes.

Waxy compounds that may be mentioned include paraffin, carnauba wax, beeswax and hydrogenated castor oil.

Among the organic solvents that may be mentioned are lower alcohols and polyols. These solvents may be chosen from glycols and glycol ethers, for instance ethylene glycol, propylene glycol, butylene glycol, dipropylene glycol or diethylene glycol.

The thickeners may be chosen especially from crosslinked acrylic polymers, for instance Carbomers, acrylate/C₁₀-C₃₀ alkylacrylate crosslinked polymers of the type such as Pemulen or polyacrylate-3 sold under the name Viscophobe DB 1000 by Amerchol; poly-acrylamides such as the emulsion of polyacrylamide, C₁₃-C₁₄ isoparaffin and laureth-7 sold under the name Sepigel 305 by SEPPIC, AMPS homopolymers or copolymers such as Hostacerin AMPS sold by Clariant, modified or unmodified guar gums and celluloses, such as hydroxy-propyl guar gum, methylhydroxyethylcellulose and hydroxypropylmethyl cellulose, xanthan gum, and nanometric silicas of the Aerosil type.

Needless to say, those skilled in the art will take care to select the optional additional compound(s) mentioned above and/or the amounts thereof such that the advantageous properties intrinsically associated with the compositions in accordance with the invention are not, or are not substantially, adversely affected by the envisaged addition(s).

The compositions according to the invention may be prepared according to techniques that are well known to those skilled in the art, in particular those intended for the preparation of emulsions of oil-in-water or water-in-oil type.

This composition may be in particular in the form of a simple or complex emulsion (O/W, W/O, O/W/O or W/O/W emulsion) such as a cream or a milk, in the form of a gel or a cream-gel, or in the form of a lotion, an oil, a powder or a solid tube, and may optionally be packaged as an aerosol and may be in the form of a mousse or a spray.

The compositions according to the invention are preferably in the form of an oil-in-water or water-in-oil emulsion.

When it is an emulsion, the aqueous phase of this emulsion may comprise a nonionic vesicular dispersion prepared according to known processes (Bangham, Standish and Watkins, J. Mol. Biol. 13, 238 (1965), FR-2,315,991 and FR-2,416,008).

When the cosmetic composition according to the invention is used to care for the human epidermis, it may be in the form of a suspension or a dispersion in solvents or fatty substances, in the form of a nonionic vesicular dispersion or in the form of an emulsion, preferably of oil-in-water type, such as a cream or a milk, or in the form of an ointment, a gel, a cream-gel, an antisun oil, a solid tube, a powder, an aerosol mousse or a spray.

When the cosmetic composition according to the invention is used for haircare, it may be in the form of a shampoo, a lotion, a gel, an emulsion or a nonionic vesicular dispersion and may constitute, for example, a rinse-out composition, to be applied before or after shampooing, before or after dyeing or bleaching, or before, during or after permanent-waving or relaxing the hair, a styling or treating lotion or gel, a blow-drying or hairsetting lotion or gel, or a composition for permanent-waving, relaxing, dyeing or bleaching the hair.

When the composition is used as a makeup product for the nails, the lips, the eyelashes, the eyebrows or the skin, such as an epidermal treatment cream, a foundation, a tube of lipstick, an eyeshadow, a makeup rouge, a mascara or an eyeliner, it may be in solid or pasty, anhydrous or aqueous form, such as oil-in-water or water-in-oil emulsions, nonionic vesicular dispersions or suspensions.

As a guide, for the antisun formulations in accordance with the invention which contain a support of oil-in-water emulsion type, the aqueous phase (especially comprising the hydrophilic screening agents) generally represents from 50% to 95% by weight and preferably from 70% to 90% by weight relative to the total weight of the formulation, the oily phase (especially comprising the lipophilic screening agents) generally represents from 5% to 50% by weight and preferably from 10% to 30% by weight relative to the total weight of the formulation, and the (co)-emulsifier(s) generally represent(s) from 0.5% to 20% by weight and preferably from 2% to 10% by weight relative to the total weight of the formulation.

As indicated at the start of the description, one subject of the invention is the use of a composition as defined above for the manufacture of a cosmetic or dermatological composition for protecting the skin and/or the hair against ultraviolet radiation and in particular solar radiation.

Another subject of the present invention lies in a process for improving the stability of at least one dibenzoylmethane derivative with respect to UV radiation, which consists in combining the said dibenzoylmethane derivative with an effective amount of at least one 3-(2-azacycloalkylidene)-1,3-dihydroindol-2-one compound as defined above.

Another subject of the present invention consists of the use of a 3-(2-azacycloalkylidene)-1,3-dihydroindol-2-one compound as defined above in the preparation of a cosmetic or dermatological composition comprising at least one UV-screening agent of the type derived from dibenzoylmethane, with the aim of improving the stability of the said dibenzoylmethane derivative with respect to UV rays.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLES

### Example 1

In this example, the photostability of 4-tert-butyl-4′-methoxydibenzoylmethane (“Parsol 1789” sunscreen from Hoffmann LaRoche) was studied in the presence of a photostabilizing compound according to the invention, namely:

1-Ethyl-3-(1-methylpyrrolidin-2-ylidene)-1,3-dihydro-indol-2-one (compound 1)

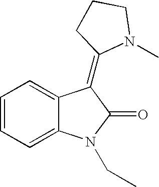

This compound may be purchased from Specs/Biospecs under the reference AH-262/33341016 or may be obtained according to the synthetic process defined above, starting with 1-ethyl-1,3-dihydroindol-2-one (CAS: 61-28-9) and 1-methylpyrrolidin-2-one (CAS: 872-50-4).

For comparative purposes, the photostability of this same screening agent was studied in the absence of this photostabilizing compound.

A solution containing 0.2% (percentage by mass) of Parsol 1789 and 0.8% of 1-ethyl-3-(1-methyl-pyrrolidin-2-ylidene)-1,3-dihydroindol-2-one (compound 1) in a mixture containing 20% Finsolv TN+80% ethanol is prepared.

After evaporation, on a support, of the ethanol contained in 10 μl of the solution described above, an oily film with a mean thickness of ˜20 μm containing 1% Parsol 1789+4% photostabilizing compound is obtained. This film is exposed to a solar simulator (19 mW/cm² of UVA and 1.2 mW/cm² of UVB) for one hour.

The compositions of these two formula (F0-F1) were thus the following (weight % relative to the total weight of the formula):

| | Photostabilizing | |
|---|---|---|
| Composition | Compound | Support |
| FO (Comparative) | 0% | Common Support* |
| F1 (Invention) | 4% | Common Support* |

*the composition of the common support was itself as follows (weight % relative to the total weight of the formula):
4-tert-butyl-4′-methoxydibenzoylmethane (Parsol 1789) 1%
C₁₂-C₁₅ alkylbenzoate (Finsolv TN) q.s. 100%

The photostability of the Parsol 1789 in these formulations was quantified by HPLC assay of the residual screening agent.

The amount of residual screening agent after irradiation is expressed mathematically by the ratio between the concentration of screening agent measured in the irradiated sample and the initial concentration of this screening agent in the sample before irradiation.

The results obtained were as follows:

| Compositions | Residual Parsol 1789 after Irradiation for 1 Hour |
|---|---|
| F0 (Comparative) | 61% |
| F1 (Invention) | 99% |

These results clearly demonstrate the noteworthy photostabilizing effect provided by 1-ethyl-3-(1-methylpyrrolidin-2-ylidene)-1,3-dihydroindol-2-one (compound 1) in accordance with the invention on 4-tert-butyl-4′-methoxydibenzoylmethane.

### Example 2

1-Ethyl-3-pyrrolidin-2-ylidene-1,3-dihydroindol-2-one (Compound 2):

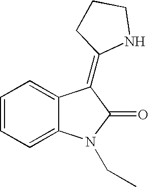

This compound may be purchased from Specs/Biospecs under the reference AH-262/33341015 or may be obtained according to the synthetic process defined above, starting with 1-ethyl-1,3-dihydroindol-2-one (CAS: 61-28-9) and 1-pyrrolidin-2-one (CAS: 616-45-5).

A photostabilizing effect on 4-tert-butyl-4′-methoxydibenzoylmethane comparable to that of Example 1 is obtained.

### Example 3

3-Pyrrolidin-2-ylidene-1,3-dihydroindol-2-one (Compound 3):

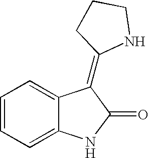

This compound may be purchased from Specs/Biospecs under the reference AH-262/33341017 or may be obtained according to the synthetic process defined above, starting with 1,3-dihydroindol-2-one (CAS: 59-48-3) and 1-pyrrolidin-2-one (CAS: 616-45-5). A photostabilizing effect on 4-tert-butyl-4′-methoxy-dibenzoylmethane comparable to that of Example 1 is obtained.

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A topically applicable cosmetic/dermatological sunscreen composition suited for the UV-photoprotection of the skin and/or hair, comprising (a) an effective UV-screening amount of at least one dibenzoylmethane compound and (b) an effective UV-photostabilizing amount of at least one 3-(2-azacycloalkylidene)-1,3-dihydroindol-2-one compound, formulated into (c) a topically applicable, cosmetically/dermatologically acceptable carrier therefor.

2. The cosmetic/dermatological composition as defined by claim 1, said at least one 3-(2-azacycloalkylidene)-1,3-dihydroindol-2-one compound having the following structural formula (I): 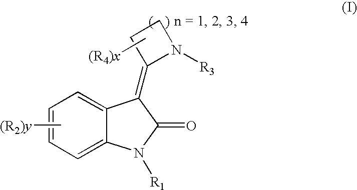
in which R₁ and R₃, which may be identical or different, are each a hydrogen atom, a saturated or unsaturated, linear or branched, C₁-C₂₂ alkyl radical optionally substituted with one or more groups A₁, a saturated or unsaturated, 4-to 7-atom ring member optionally containing at least one heteroatom selected from among N, O and S, optionally fused with another ring, optionally substituted with one or more groups A₁, with the proviso that these rings may contain one or more carbonyl or thiocarbonyl functional groups, one of the groups C(═NR₅)R′₅, C(═NR₅)NR′₅R″₅, COR₅, CSR₅, COOR₅, CONR₅R′₅, CSNR₅R′₅, SO₂R₅, and SO₂NR₅R′₅ wherein R₅, R′₅ and R″₅, which may be identical or different, are each hydrogen, a linear or branched C₁-C₂₂ alkyl radical or a 4- to 7-atom ring member optionally containing at least one heteroatom selected from among N, O, S, optionally fused with another ring, with the proviso that these rings may contain one or more carbonyl or thiocarbonyl functional groups, the alkyl radical of said rings being saturated or unsaturated and optionally substituted with at least one substituent A₂; R₂ and R₄, which may be identical or different, are each a hydrogen atom, a saturated or unsaturated, linear or branched C₁-C₂₂ alkyl radical optionally substituted with one or more groups A₁, a saturated or unsaturated, 4- to 7-atom ring member optionally containing at least one heteroatom selected from among N, O and S, optionally substituted with one or more groups A₁, optionally fused with another ring or with another 3-(2-azacycloalkylidene)-1,3-dihydroindol-2-one compound, with the proviso that these rings may contain one or more carbonyl or thiocarbonyl functional groups, optionally positively charged, a halogen, one of the groups CF₃, CN, OR₅, SR₅, NR₅R′₅, C(═NR₅)R′₅, C(═NR₅)NR′₅R″₅, NR₅C(═NR′₅)NR″₅R′″₅, COR₅, CSR₅, COOR₅, CONR₅R′₅, NR₅COR′₅, NR₅CONR′₅R″₅, CSNR₅R′₅, SO₂NR₅R′₅, NR₅SO₂R′₅, SO₂R₅, and NR₅R′₅″R₅R′″₅⁺ wherein R₅, R′₅, R″₅ and R′″5, which may be identical or different, are each hydrogen, a linear or branched C₁-C₂₂ alkyl radical or a 4- to 7-atom ring member which may contain at least one heteroatom selected from among N, O, S, optionally fused with another ring, with the proviso that these rings may contain one or more carbonyl or thiocarbonyl functional groups, the alkyl radical or the said rings being saturated or unsaturated and optionally substituted with at least one substituent A₂; A₁ is a halogen, a saturated or unsaturated, linear or branched C₁-C₂₂ alkyl radical, optionally substituted with one or more groups A₂, one of the groups CF₃, CN, OR₅, SR₅, NR₅R′₅, C(═NR₅)R′₅, C(═NR₅)NR′₅R″₅, NR₅C(═NR′₅)NR″₅R′″₅, COR₅, CSR₅, COOR₅, CONR₅R′₅, NR₅COR′₅, NR₅CONR′₅R″₅, CSNR₅R′₅, SO₂NR₅R′₅, NR₅SO₂R′₅, SO₂R₅, SiR₅R′₅R″₅, SiR₅(OsiR′₅R″₅R′″₅)OSiR′₅R″₅R′″₅, and NR₅R′₅R″₅R′″₅+ wherein R₅, R′₅, R″₅ and R′″₅, which may be identical or different, are each hydrogen, a linear or branched C₁-C₂₂ alkyl radical or a 4- to 7-atom ring member which may contain at least one heteroatom selected from among N, O, S, optionally fused with another ring, with the proviso that these rings may contain one or more carbonyl or thiocarbonyl functional groups, the alkyl radical or said rings being saturated or unsaturated and optionally substituted with at least one substituent A₂, a saturated or unsaturated 4- to 7-atom ring member optionally containing at least one heteroatom selected from among N, O and S, optionally substituted with one or more groups A₂, optionally fused with another ring or with another 3-(2-azacycloalkylidene)-1,3-dihydroindol-2-one compound, with the proviso that these rings may contain one or more carbonyl or thiocarbonyl functional groups, optionally positively charged; A₂ is a halogen, a saturated or unsaturated, linear or branched C₁-C₂₂ alkyl radical, one of the groups CF₃, CN, OR, SR, NRR′, C(═NR)R′, C(═NR)NR′R″, NRC(═NR′)NR″R′″, COR, CSR, COOR, CONRR′, NRCOR′, NRCONR′R″, CSNRR′, SO₂NRR′, NRSO₂R′, SO₂R, SiRR′R″, SiR(OSiR′R″R′″)OSiR′R″R′″, and NRR′R″R′″+ wherein R, R′, R″ and R′″, which may be identical or different, are each hydrogen or a linear or branched C₁-C₂₂ alkyl radical; y is 1, 2, 3 or 4; and x ranges from 1 to 2n+2, formulated into (b) a topically applicable, cosmetically/dermatologically acceptable carrier therefor.

3. The cosmetic/dermatological composition as defined by claim 2, said at least one 3-(2-azacycloalkylidene)-1,3-dihydroindol-2-one compound having one of the following formulae (II) or (III): 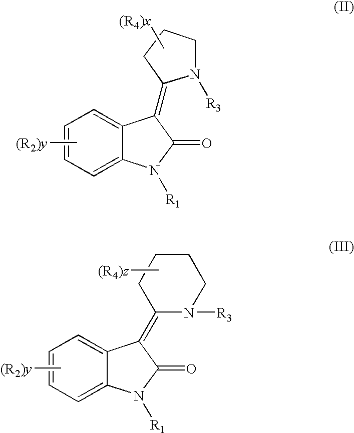
in which R₁ and R₃, which may be identical or different, are each hydrogen, a saturated or unsaturated, linear or branched C₁-C₈ alkyl radical, a phenyl radical, or a radical COR₅ or SO₂R₅ wherein R₅ is a saturated or unsaturated, linear or branched C₁-C₈ alkyl radical, or a phenyl radical; R₂ is hydrogen, a saturated or unsaturated, linear or branched C₁-C₈ alkyl radical, a phenyl radical, one of the radicals OR₅, NR₅′₅, NR₅COR′₅, COOR₅ and CONR₅R′₅ wherein R₅ and R′₅, which may be identical or different, are each hydrogen, a saturated or unsaturated, linear or branched C₁-C₈ alkyl radical, a phenyl radical, or the radical CF₃; R₄ is hydrogen, a saturated or unsaturated, linear or branched C₁-C₈ alkyl radical, a phenyl radical or a radical OR₅ wherein R₅ is hydrogen, a saturated or unsaturated, linear or branched C₁-C₈ alkyl radical, or a phenyl radical; and x, y and z are each 1, 2, 3 or 4.

4. The cosmetic/dermatological composition as defined by claim 3, comprising at least one compound of formula (II), wherein: R₂ is hydrogen, OR₅, NR₅COR′₅ or NR₅R′₅ wherein R₅ and R′₅, which may be identical or different, are each hydrogen, a saturated or unsaturated, linear or branched C₁-C₈ alkyl radical, or a phenyl radical; R₄ is hydrogen; and R₁ and R₃, which may be identical or different, are each hydrogen, a saturated or unsaturated, linear or branched C₁-C₈ alkyl radical, or a phenyl radical.

5. The cosmetic/dermatological composition as defined by claim 3, comprising at least one compound of formula (II) selected from the group consisting of 1-ethyl-3-(1-methylpyrrolidin-2-ylidene)-1,3-dihydroindol-2-one 1-ethyl-3-pyrrolidin-2-ylidene-1,3-dihydroindol-2-one and pyrrolidine-2-ylidene-1,3-dihydroindol-2-one.

6. The cosmetic/dermatological composition as defined by claim 1, said at least one 3-(2-azacycloalkylidene)-1,3-dihydroindol-2-one compound comprising from 0.01% to 20% by weight thereof.

7. The cosmetic/dermatological composition as defined by claim 1, said at least one 3-(2-azacycloalkylidene)-1,3-dihydroindol-2-one compound comprising from 0.1% to 10% by weight thereof.

8. The cosmetic/dermatological composition as defined by claim 1, said at least dibenzoylmethane compound being selected from the group consisting of:
2-methyldibenzoylmethane,
4-methyldibenzoylmethane,
4-isopropyldibenzoylmethane,
4-tert-butyldibenzoylmethane,
2,4-dimethyldibenzoylmethane,
2,5-dimethyldibenzoylmethane,
4,4′-diisopropyldibenzoylmethane,
4,4′-dimethoxydibenzoylmethane,
4-tert-butyl-4′-methoxydibenzoylmethane,
2-methyl-5-isopropyl-4′-methoxydibenzoylmethane,
2-methyl-5-tert-butyl-4′-methoxydibenzoylmethane,
2,4-dimethyl-4′-methoxydibenzoylmethane,
2,6-dimethyl-4-tert-butyl-4′-methoxydibenzoylmethane, and mixtures thereof.

9. The cosmetic/dermatological composition as defined by claim 8, said at least dibenzoylmethane compound comprising 4-tert-butyl-4′-methoxydibenzoylmethane.

10. The cosmetic/dermatological composition as defined by claim 8, said at least dibenzoylmethane compound comprising 4-isopropyldibenzoylmethane.

11. The cosmetic/dermatological composition as defined by claim 1, said at least dibenzoylmethane compound comprising from 0.01% to 20% by weight thereof.

12. The cosmetic/dermatological composition as defined by claim 1, said at least dibenzoylmethane compound comprising from 0.1% to 10% by weight thereof.

13. The cosmetic/dermatological composition as defined by claim 1, formulated as an oil-in-water or water-in-oil emulsion.

14. The cosmetic/dermatological composition as defined by claim 1, further comprising at least one additional organic and/or inorganic UV-A and/or UV-B sunscreen.

15. The cosmetic/dermatological composition as defined by claim 14, comprising at least one additional organic sunscreen selected from the group consisting of anthranilates; cinnamic derivatives; dibenzoylmethane derivatives; salicylic derivatives, camphor derivatives; triazine derivatives; benzophenone derivatives; β,β-diphenyl acrylate derivatives; benzotriazole derivatives; benzalmalonate derivatives; benzimidazole derivatives; imidazolines; bis-benzoazolyl derivatives; p-aminobenzoic acid (PABA) derivatives; methylenebis(hydroxyphenylbenzotriazole) derivatives; benzoxazole derivatives; screening polymers and screening silicones; dimers derived from α-alkylstyrene; 4,4-diarylbutadiene derivatives, and mixtures thereof.

16. The cosmetic/dermatological composition as defined by claim 15, said at least one additional organic sunscreen being selected from the group consisting of:
Ethylhexyl Salicylate,
Ethylhexyl Methoxycinnamate, Octocrylene,
Phenylbenzimidazole Sulphonic Acid,
Benzophenone-3,
Benzophenone-4,
Benzophenone-5,
n-Hexyl 2-(4-diethylamino-2-hydroxybenzoyl)benzoate,
4-Methylbenzylidene camphor,
Terephthalylidene dicamphor sulfonic acid,
Disodium Phenyl Dibenzimidazole Tetra-sulphonate,
2,4,6-Tris(diisobutyl 4′-aminobenzalmalonate)-s-triazine,
Anisotriazine,
Ethylhexyl triazone,
Diethylhexyl Butamido Triazone,
Methylene bis-Benzotriazolyl Tetramethylbutylphenol,
Drometrizole Trisiloxane,
Polysilicone-15
1,1-dicarboxy(2,2′-dimethylpropyl)-4,4-diphenylbutadiene
2,4-bis-[5-1 (dimethylpropyl)benzoxazol-2-yl-(4-phenyl)-imino]-6-(2-ethylhexyl)-imino-1,3,5-triazine, and mixtures thereof.

17. The cosmetic/dermatological composition as defined by claim 14, comprising at least one additional inorganic sunscreen pigment or nanopigment of a metal oxide, coated or uncoated.

18. The cosmetic/dermatological composition as defined by claim 17, said at least one pigment or nanopigment comprising titanium, zinc, iron, zirconium and/or cerium oxides, and mixtures thereof, coated or uncoated.

19. The cosmetic/dermatological composition as defined by claim 1, further comprising at least one agent for artificially bronzing and/or tanning the skin.

20. The cosmetic/dermatological composition as defined by claim 1, further comprising at least one adjuvant selected from the group consisting of fatty substances, organic solvents, ionic or nonionic thickeners, demulcents, humectants, antioxidants, moisturizers, desquamating agents, anti-free-radical agents, antipollution agents, antibacterials, anti-inflammatory agents, depigmenting agents, propigmenting agents, opacifiers, stabilizers, emollients, silicones, anti-foaming agents, insect repellents, perfumes, preservatives, anionic, cationic, nonionic, zwitterionic or amphoteric surfactants, substance P antagonists, substance CGRP antagonists, fillers, pigments, polymers, propellants, alkalinizing or acidifying agents, and mixtures thereof.

21. The cosmetic/dermatological composition as defined by claim 1, formulated as a nonionic vesicular dispersion, a cream, a milk, a gel, a gel cream, a suspension, a dispersion, an oil, a powder, a solid stick, a mousse or a spray.

22. The cosmetic/dermatological composition as defined by claim 1, formulated as a makeup.

23. The cosmetic/dermatological composition as defined by claim 1, formulated for protecting the hair against ultraviolet rays and in the form of a shampoo, a lotion, a gel, an emulsion or a nonionic vesicular dispersion.

24. A regime or regimen for photoprotecting the skin and/or hair against the damaging effects of UV radiation, comprising topically applying thereon an effective amount of the cosmetic/dermatological composition as defined by claim 1.
